(19) United States
(12) Patent Application Publication
(10) Pub. No.: US 2007/0134190 A1
(43) Pub. Date: Jun. 14, 2007

(54) All weather hair conditioning composition

(75) Inventors: David W. Cannell, Plainfield, NJ (US); Mike Wong, Plainsboro, NJ (US)

Correspondence Address: L'OREAL USA/ PATENT DEPARTMENT, 30 TERMINAL AVENUE, CLARK, NJ, 07066, US

(73) Assignee: L'OREAL, Paris (FR)

(21) Appl. No.: 11/301,184

(22) Filed: Dec. 12, 2005

(51) Int. Cl. A61K 8/73; A61K 8/55

(52) U.S. Cl. 424/70.17; 424/70.7; 424/70.23

(57) ABSTRACT

A cosmetic composition capable of inhibiting hair from losing water containing: at least one lecithin; at least one amphoteric surfactant; at least one nonionic surfactant; at least one film forming polymer; at least one cationic polymer; and at least one small molecule capable of inhibiting water loss from hair fibers when they are exposed to high or low humidity.

### BACKGROUND OF THE INVENTION

Hair fibers, depending on whether they are exposed to low or highly humid conditions, have a tendency to lose their shape, curl definition and/or become frizzy. These problems are the result of water loss from the fibers. In an effort to solve such problems, hair benefit agents such as styling polymers are oftentimes incorporated into rinse-off hair products (shampoos, conditioners and the like) in order to seal in moisture within the hair fibers, thereby inhibiting water loss therefrom. Unfortunately, these hair benefit agents are either rinsed off and/or neutralized after their application onto the hair fibers.

It is therefore an object of the present invention to provide a composition and process for inhibiting water loss from hair fibers upon exposure to high or low humidity.

### SUMMARY OF THE INVENTION

The present invention is directed to the surprising discovery that a hair treatment composition capable of inhibiting water loss from hair fibers upon exposure to both high and low humidity can be prepared by combining:
(a) at least one lecithin;
(b) at least one amphoteric surfactant;
(c) at least one nonionic surfactant;
(d) at least one film forming polymer;
(e) at least one cationic polymer; and
(f) at least one polar amino compound.
   The first three components listed (i.e., at least one lecithin, at least one amphoteric surfactant, at least one nonionic surfactant) are sometimes referred to as a “LAN system” or simply as “LAN” herein. Preferably, the film forming polymer is a non-neutralized resin.

### DETAILED DESCRIPTION OF THE PRESENT INVENTION

Other than in the operating examples, or where otherwise indicated, all numbers expressing quantities of ingredients and/or reaction conditions, are to be understood as being modified in all instances by the term “about”.

Lecithins are mixtures of phospholipids, i.e., of diglycerides of fatty acids linked to an ester of phosphoric acid. Preferably, lecithins are diglycerides of stearic, palmitic, and oleic acids linked to the choline ester of phosphoric acid. Lecithin is usually defined either as pure phosphatidyl cholines or as crude mixtures of phospholipids which include phosphatidyl choline, phosphatidyl serine, phosphatidyl ethanolamine, phosphatidyl inositol, other phospholipids, and a variety of other compounds such as fatty acids, triglycerides, sterols, carbohydrates, and glycolipids.

The lecithin used in the present invention include the types described above and may be present in the form of a liquid, powder, or granules. Lecithins useful in the invention include, but are not limited to, soy lecithin and hydroxylated lecithin. For example, ALCOLEC S is a fluid soy lecithin, ALCOLEC F 100 is a powder soy lecithin, and ALCOLEC Z3 is a hydroxylated lecithin, all of which are available from the American Lecithin Company.

In the present invention, lecithin is used in an amount of from greater than 0 to about 5% by weight, based on the weight of the composition as a whole. Since lecithin itself is not necessarily a pure raw material and may have free glycerides, glycerin, fatty acids, and soaps, adjustments in this amount may need to be made, i.e., one source of lecithin may require different ratios of nonionic and amphoteric surfactants than another to achieve, e.g., maximum clarity of solution. Preferably, the composition of the invention forms a clear solution, though the purpose of the invention is achieved just as effectively with a cloudy or slightly cloudy solution, etc.

A group of phospholipids which can be used in the present invention as lecithins are multifunctional biomimetic phospholipids, including, for example, the following multifunctional biomimetic phospholipids manufactured by Mona Industries: PHOSPHOLIPID PTC, PHOSPHOLIPID CDM, PHOSPHOLIPID SV, PHOSPHOLIPID GLA, and PHOSPHOLIPID EFA.

The amphoteric surfactants useful in the present invention include, but are not limited to, betaines, sultaines, hydroxysultaines, alkyl amphodiacetates, alkyl amphodipropionates, and imidazolines, or salts thereof. It is recognized that other fatty acid condensates such as those formed with amino acids, proteins, and the like are suitable. Cocamphodipropionate is particularly preferred, for example, MIRANOL C2M-SF Conc. (disodium cocamphodipropionate), in its salt-free form, available from Rhône-Poulenc. Also preferred is CROSULTAINE C-50 (cocamidopropyl hydroxysultaine), available from Croda.

The amphoteric surfactants are typically present in an amount of from greater than 0% to 10% by weight, based on the weight of the total composition. Preferably, the amphoteric surfactant is present in the composition in an amount ranging from 2 to 10% by weight of the composition as a whole, when 5% of the lecithin is used. When the lecithin/amphoteric/nonionic system is employed as a carrier for a water-insoluble polymer or resin, the amphoteric surfactants are preferably present in the composition in an amount ranging from 6 to 10% by weight. When the lecithin/amphoteric/nonionic system is employed as a carrier for a lipophilic material, the amphoteric surfactants are preferably present in the composition in an amount ranging from 4 to 8% by weight. Other amphoteric surfactants useful in the present invention include disodium wheatgermimido PEG-2 sulfosuccinate, available under the trade name MACKANATE WGD from McIntyre Group Ltd. and disodium soyamphodiacetate, available under the trade name MACKAM 2S from McIntyre Group Ltd.The nonionic surfactants useful in the present invention are preferably formed from a fatty alcohol, a fatty acid, or a glyceride with a C₈ to C₂₄ carbon chain, preferably a C₁₂ to C₁₈ carbon chain, more preferably a C₁₆ to C₁₈ carbon chain, derivatized to yield a Hydrophilic-Lipophilic Balance (HLB) of at least 10. HLB is understood to mean the balance between the size and strength of the hydrophilic group and the size and strength of the lipophilic group of the surfactant. Such derivatives can be polymers such as ethoxylates, propoxylates, polyglucosides, polyglycerins, polylactates, polyglycolates, polysorbates, and others that would be apparent to one of ordinary skill in the art. Such derivatives may also be mixed polymers of the above, such as ethoxylate/propoxylate species, where the total HLB is preferably greater than or equal to 10. Preferably the nonionic surfactants contain ethoxylate in a molar content of from 10-25, more preferably from 10-20 moles.

Nonionic surfactants may be selected from, but are not limited to, the following:

| #of Cs | Name | Trade Name |
|---|---|---|
| C-12 | Laureth-23 | BRIJ 35, available from ICI Surfactants |
| C-16 | Ceteth-10 | BRIJ 56, available from ICI Surfactants |
| C-16 | Ceteth-20 | BRIJ 58, available from ICI Surfactants |
| C-16 | IsoCeteth-20 | Arlasolve 200, available from ICI Surfactants |
| C-18 | Steareth-10 | Volpo S-10, available from Croda Chemicals Ltd. |
| C-18 | Steareth-16 | Solulan-16, available from Amerchol Corp. |
| C-18 | Steareth-20 | BRIJ 78, available from ICI Surfactants |
| C-18 | Steareth-25 | Solulan-25, available from Amerchol Corp. |
| C-18= | Oleth-10 | BRIJ 97, available from ICI Surfactants |
| C-18= | Oleth-20 | Volpo-20, available from Croda Chemicals Ltd. |

Alkyl polyglucose surfactants sold under the name PLANTAREN, available from Henkel, may also be used. The nonionic surfactant is typically employed in an amount of from greater than 0% to 20% by weight, based on the weight of the total composition. Preferably, the nonionic surfactant is present in an amount of from 5 to 20% by weight, based on the weight of the composition, when 5% lecithin is used. More preferably, the nonionic surfactant is present in an amount of from 10 to 20% by weight, based on the weight of the composition.

In one preferred embodiment of the composition of the present invention, within the LAN in particular, the lecithin, the amphoteric surfactant, and the nonionic surfactant are present in the composition such that the nonionic surfactant and the amphoteric surfactant are each present in an amount by weight greater than the amount of lecithin. In a more preferred embodiment, the amount of lecithin in the composition is kept fixed while the amounts of the amphoteric and nonionic surfactants are increased. In a still more preferred embodiment, calculating the lecithin as present at a value of 1, the phospholipid, amphoteric surfactant and nonionic surfactant are preferably present in the composition in a ratio ranging from 1/0.8/2 and above by weight relative to the whole composition, i.e., where the amounts of the surfactants can be increased independently of each other but the amount of lecithin stays fixed. The ratio is considered to be “above” 1/0.8/2 when the amount of either of the surfactants increases. Another preferable range is from 1/1.2/2 and above. A further preferred ratio is 1/1.2/3 and above, and more preferably above 1/1.2/4. The loading capability for hydrophobes carried by the LAN system of the present invention is maximized if the ratio of nonionic surfactant to lecithin is minimized, with bilayers formed by the lecithin still being solubilized, because an excess of nonionic surfactant may disrupt the organized structure.

In one preferred embodiment, the composition of the present invention comprises ALCOLEC S (soy lecithin), MIRANOL C2M-SF Conc. (disodium cocamphodipropionate, an amphoteric surfactant), ARLASOLVE 200 (IsoCeteth-20, a nonionic surfactant) in a ratio of 5/6/10 (1:1.2:2) and 5/6/20 (1:1.2:4) wherein the ratios are calculated by weight relative to the whole composition. Typically, LAN compositions of the invention can resist storage at 45° C for three months or more, which would predict that they have a shelf life at room temperature of at least three years.

Film forming polymers useful herein are non-neutralized or partially neutralized, preferably non-neutralized, polymers and resins, most preferably non-neutralized resins, wherein the polymers and resins include but are not limited to those containing carboxyl moieties., such as acrylates and other carboxy polymers. Typically, water-insoluble polymers and resins have to be neutralized to about 90% of their carboxyl moieties to make them water soluble for the purpose of formulating products in aqueous solution and for the purpose of making products which have good non-build-up properties, i.e., can be easily washed off the hair after use. However, when used with the compositions of the present invention complete, some (e.g., up to 90%) or no neutralization is needed to effectively use these polymers/resins. It is believed that the combination of the lecithin, the nonionic surfactant, and the amphoteric surfactant of the present invention provides the usefulness of the water-insoluble polymers or resins.

The following are examples of film forming polymers that can be incorporated into the compositions of the present invention. The list is not intended to be limiting:
AMPHOMER LV-71 from National Starch (octylacrylamide/acrylates/butylaminoethyl methacrylate copolymer),
OMNIREZ-2000 from ISP (PVM/MA half ethyl ester copolymer),
RESYN 28-2930 from National Starch (Vinyl acetate/crotonates/vinyl neodecanoate copolymer),
LUVIMER 100P from BASF (t-butyl acrylate/ethyl acrylate/methacrylic acid), and
ULTRAHOLD STRONG from BASF (acrylic acid/ethyl acrylate/t-butyl acrylamide).
   Unneutralized or partially neutralized water-insoluble latexes can also be used as invention film-forming polymers. Included are the following latexes:
AMERHOLD DR-25 from Amerchol (acrylic acid/methacrylic acid/acrylates/methacrylates),
LUVIMER 36D from BASF (ethyl acrylate/t-butyl acrylate/methacrylic acid), and
ACUDYNE 258 from Rohm & Haas (acrylic acid/methacrylic acid/acrylates/methacrylates/hydroxy ester acrylates).

The film forming polymer is typically present in an amount ranging from greater than 0% to 15% by weight, preferably from 0.5 to 10% by weight, and more preferably from 1 to 5% by weight, based on the total weight of the composition. The concentrations of LAN and film forming polymer can be adjusted by one of ordinary skill in view of this disclosure, as can neutralization extent.

Cationic polymers useful herein include polyquaternium 4, polyquaternium 6, polyquaternium 7, polyquaternium 10, polyquaternium 11, polyquaternium 16, polyquaternium 22, and polyquaternium 32. Cationic polymers useful in the present invention include, but are not limited to, polyquaternium 4, polyquaternium 6, polyquaternium 7, polyquaternium 10, polyquaternium 11, polyquaternium 16, polyquaternium 22, polyquaternium 28, polyquaternium 32, and guar hydroxypropyltrimonium chloride. Preferred cationic polymers include POLYMER JR-125, POLYMER JR-400, Polymer JR-30 M hydroxyethyl cellulosic polymers (polyquaternium 10) available from AMERCHOL; JAGUAR C13-S, guar hydroxypropyltrimonium chloride, available from Meyhall; and MERQUAT 100 and 280, a dimethyl dialkyl ammonium chloride (polyquaternium 6) available from CALGON.

The cationic polymer is generally present in an amount of from greater than 0% to 15%, preferably from 0.5 to 10% by weight, and more preferably from 1 to 5% by weight, based on the total weight of the composition. Amounts of cationic polymer and other invention components can be adjusted relative to one another by one of ordinary skill in view of this disclosure.

Suitable small molecules for use in the present invention are those having the ability to both penetrate keratin fibers and help prevent and/or slow down water loss therefrom. Examples thereof include, but are not limited to, polar amino acids and their salts/derivatives, urea and its salts/derivatives, guanidine and its salts/derivatives, and combinations thereof.

Polar amino acids may be chosen from arginine, asparagine, aspartic acid (or aspartate), glutamine, glutamic acid (or glutamate), histidine, lysine, serine, and threonine. These amino acids are hydrophilic due to their polar side chains. Lysine and arginine are positively charged at neutral pH, whereas histidine can be uncharged or positively charged depending on its local environment.

Alternatively, proteins, polypeptides or other natural extracts having a high polar amino acid content can be used. For example, proteins having a major proportion of arginine units (in the range from about 50 to about 90%, by weight, of the total protein) in their structures are members of that class of proteins known as protamines. The protamine proteins are characterised by having: (a) a low molecular weight, in the range of about 5,000; (b) a high isoelectric point, in the pH range of about 10 to 12; and (c) a high arginine content, in the range from about 50 to about 90%, by weight of the total protein. Suitable examples are described in U.S. Pat. No. 3,997,659.

Proteins of high polar amino acid content as described above can be subjected to acid or base hydrolysis to yield polypeptides which also have a high polar amino acid content. Examples of suitable polypeptides are also described in U.S. Pat. No. 3,997,659, being protamine-derived polypeptides having a molecular weight below about 5,000, a basic pH (10-12), and an arginine content of about 50%, or greater, by weight of the total polypeptide.

Not only may naturally occurring proteins be used, but also synthetic proteins, for example, polylysine and polyarginine, or mixtures thereof.

An example of a suitable natural extract which is rich in arginine is aloe vera extract.

The polar amino acids and the proteins and polypeptides having a polar amino acid content of 50%, or greater, are often isolated from natural sources in the form of salts and hydrosalts, which are also suitable for use according to the invention. Such salts and hydrosalts are formed by reaction with mineral acids such as hydrochloric acid, phosphoric acid, carbonic acid, sulfuric acid, nitric acid, and the like, or the organic acids such as formic acid, acetic acid, lauric acid, chloroacetic acid and the like. A suitable example is arginine hydrochloride.

The most preferred small molecules for use in the present invention are arginine and urea, as well as their respective salts and/or hydrosalts.

The amount of amino compound typically employed in the present invention will range from greater than 0% to 10% by weight, preferably from .01 to 5% by weigh, and more preferably from 0.1 to 1% by weight, based on total weight of the composition.

The composition preferably also contains water and can, in addition, contain ingredients such as silicones, anionic surfactants, organic salts, inorganic salts, proteins, hair dyes, water-soluble polymers, quaternary ammonium compounds, complex and simple carbohydrates, preservatives and fragrances.

The composition of the invention preferably has a pH ranging from 2-12, preferably from 4 to 10, and more preferably from 5 to 8.

Another embodiment of the present invention is drawn to a process for inhibiting hair fibers from losing water, in general, and especially when exposed to low or high humidity. The process involves contacting the hair fibers with the above-described composition.

The present invention will be better understood by the examples which follow, all of which are intended for illustrative purposes only, and are not meant to unduly limit the scope of the invention in any way.

### EXAMPLES

The following Examples are intended for illustrative purposes only, and are not meant to unduly limit the scope of the invention in any way.

| | | Inventive |
|---|---|---|
| | Control | Shampoo |
| INCI Name | Shampoo | Composition |
| SODIUM LAURETH SULFATE | 12 | 13.85 |
| LAURETH-12 | 0.25 | |
| COCO-BETAINE | 10 | |
| COCAMIDE MEA | 0.5 | |
| COCAMIDOPROPYL BETAINE | | 5 |
| PPG-5-CETETH-20 | | 3.1 |
| DISODIUM | | 4.5 |
| COCOAMPHODIPROPIONATE | | |
| HEXYLENE GLYCOL | 1.25 | 1 |
| POLYQUATERNIUM-10 | | 1.5 |
| OCTYLACRYLAMIDE/ | | 1.5 |
| ACRYLATES/ | | |
| BUTYLAMINOETHYL | | |
| METHACRYLATE | | |
| COPOLYMER | | |
| LECITHIN | | 0.1 |
| GLUTAMIC ACID | | 1 |
| PRESERVATIVES | 0.4 | 0.55 |
| SODIUM CHLORIDE | 1 | |
| WATER | q.s. TO 100% | q.s. TO 100% |

For curl retention, hair tresses treated with either the control shampoo or the inventive shampoo composition were put into a chamber at low humidity (26%) for 6 hours and readings were taken at 0, 5 mins, 15 mins, 30 mins, 1 hour, 2 hours, 4 hours and 6 hours. The tresses were then left at ambient humidity overnight before increasing the humidity to 79%. Same time points were recorded.

Results

Curl Retention at Low Humidity (26%)

It was found that the percent curl retention decreased over time for both the control and the inventive shampoo compositions. However, those tresses treated with the inventive shampoo were observed to have a statistically significantly greater percentage of curl retention as compared to tresses treated with the control shampoo.

Curl Retention at High Humidity (79%)

It was found that the percent curl retention decreased over time for both the control and the inventive shampoo compositions. However, those tresses treated with the inventive shampoo were observed to have a statistically significantly greater percentage of curl retention as compared to tresses treated with the control shampoo.

Frizz Measurement at Low Humidity (26%)

For frizz control, hair tresses treated with either the control shampoo or the inventive shampoo composition were put into the chamber at low humidity (26%) for 6 hours and readings were taken at 0, 5 mins, 15 mins, 30 mins, 1 hour, 2 hours, 4 hours and 6 hours. The tresses were then left at ambient humidity overnight. The humidity was then increased to 79%. Same time points were recorded.

It was observed that hair tresses treated with the inventive shampoo composition exhibited statistically significantly lower frizz (i.e., had a smaller frizz area) as compared to those tresses treated with the control shampoo.

Thus, it can be seen that hair treated with a composition in accordance with the present invention possesses increased curl retention and frizz control properties due to the hair's ability to inhibit water loss.

What is claimed is:

1. A cosmetic composition capable of inhibiting water loss from hair fibers comprising:
(a) at least one lecithin;
(b) at least one amphoteric surfactant;
(c) at least one nonionic surfactant;
(d) at least one film forming polymer;
(e) at least one cationic polymer; and
(f) at least one small molecule.

2. The composition of claim 1 wherein the at least one lecithin is employed in an amount of from greater than 0% to about 5% by weight, based on the weight of the composition.

3. The composition of claim 1 wherein the at least one amphoteric surfactant is employed in an amount of from greater than 0% to about 10% by weight, based on the weight of the composition.

4. The composition of claim 1 wherein the at least one nonionic surfactant is employed in an amount of from greater than 0% to about 20% by weight, based on the weight of the composition.

5. The composition of claim 1 wherein the ratio by weight of (a):(b):(c) is about 1:0.8:2.

6. The composition of claim 1 wherein the ratio by weight of (a):(b):(c) is about 1:1.2:2.

7. The composition of claim 1 wherein the ratio by weight of (a):(b):(c) is about 1:1.2:4.

8. The composition of claim 1 wherein the at least one film-forming polymer is employed in an amount of from greater than 0% to about 15% by weight, based on the weight of the composition.

9. The composition of claim 1 wherein the at least one cationic polymer is employed in an amount of from greater than 0% to about 15% by weight, based on the weight of the composition.

10. The composition of claim 1 wherein the at least one small molecule is chosen from polar amino acids, their salts and derivatives; urea, its salts and derivatives; and guanidine, its salts and derivatives.

11. The composition of claim 1 wherein the at least one small molecule is chosen from polar amino acids.

12. The composition of claim 1 wherein the at least one polar amino compound is present in an amount of from greater than 0% by weight to about 10% by weight, based on the weight of the composition.

13. The composition of claim 1 wherein the composition has a pH ranging from about 5 to about 8.

14. A process for inhibiting hair fibers from losing water when exposed to high or low humidity comprising contacting the hair fibers with the composition of claim 1.
